(19) United States
(12) Patent Application Publication
(10) Pub. No.: US 2025/0248725 A1
(43) Pub. Date: Aug. 7, 2025

(54) Endoscopic Tool

(71) Applicant: NMGN MEDICAL, Wijchen (NL)

(72) Inventors: Antonius Maria Brouwers, Wijchen (NL); Mark Paul Lamberts, Nijmegen (NL)

(21) Appl. No.: 19/112,519

(22) PCT Filed: Jun. 13, 2023
(86) PCT No.: PCT/NL2023/050328

§ 371 Date: Mar. 17, 2025

(30) Foreign Application Priority Data
Jun. 13, 2022 (NL) 2032146
Jun. 13, 2023 (WO) PCT/NL23/50328

(51) Int. Cl. A61B 17/221; A61B 1/018; A61B 1/04; A61B 17/00

(52) U.S. Cl. CPC A61B 17/221; A61B 1/018; A61B 1/042; A61B 17/00234; A61B 2017/00238; A61B 2017/00296; A61B 2017/2212

(57) ABSTRACT

Devices and methods which allow a foreign object, in particular an elongated foreign object, to be removed through a narrow passage, in particular with reduced chance of getting stuck inside said narrow passage. It will be generally understood that devices and methods disclosed herein may be applied in a medical field, but also in a non-medical field.

### TECHNICAL FIELD

The aspects and embodiments thereof relate to the field of removing objects from narrow passages, for example using an endoscopic tool.

### BACKGROUND

In the medical field, endoscopic foreign object retrieval methods are used to for example remove ingested foreign objects from the gastro-intestinal tract or the respiratory tract of a human or animal. The methods are minimally invasive, as they typically do not involve surgery. Instead, an endoscopic tool is inserted into a body passage of the human or animal via an orifice such as the mouth or anus. To provide a view of the endoscopic tool inside the body passage, an endoscopic camera may be used.

The endoscopic camera is typically shaped as an elongated tube, and is typically provided with an optic fibre camera which allows an operator to view inside the passage into which the endoscopic tool has been inserted. The endoscopic camera may be provided with a working channel into which the endoscopic tool may be inserted. As such, the endoscopic tool may be inserted into the body passage via the working channel of the endoscopic camera. In other words, the working channel of the endoscopic camera is used to guide the endoscopic tool inside the body passage.

For removing the foreign object, the endoscopic tool further comprises a manipulation tool arranged for grasping the object. After the object has been grasped, the endoscopic tool is retracted out of the passage, together with the object.

In the non-medical field, endoscopic cameras are used to inspect narrow passages, such as plumbing or pipes. When a foreign object is detected in such plumbing or pipes, a manipulation tool of an endoscopic tool may be used to grasp said foreign object, and the foreign object may as such be removed from the plumbing or pipes together with the endoscopic tool.

### SUMMARY

It has been observed that foreign objects which have to be removed using an endoscopic tool may be elongated foreign objects which have a length exceeding a diameter of the passage through which the objects have to be retrieved. Examples of such objects are spoons, toothpicks, toothbrushes, and batteries. It has been further observed that these elongated foreign objects are prone to getting stuck in the passage through which they have to be retrieved due to their length.

It is an object of the present disclosure to provide devices and methods which allow a foreign object, in particular an elongated foreign object, to be removed through a narrow passage, in particular with reduced chance of getting stuck inside said narrow passage. It will be generally understood that devices and methods disclosed herein may be applied in a medical field, but also in a non-medical field.

A first aspect provides a device for retrieving a foreign object through an elongated passage. The device comprises a first object manipulation tool, a second object manipulation tool, and an elongated housing, such as a flexible tube, arranged for accommodating at least part of the first object manipulation tool, and preferably also at least part of the second object manipulation tool.

The first object manipulation tool, and preferably also the second object manipulation tool, is arranged to be partially moved out of the housing at or near a distal end of the housing, and the first object manipulation tool is arranged to have a distal end of the first object manipulation tool positioned alongside and preferably outside the housing at a distance from the distal end of the housing. When the distal end of the first object manipulation tool can be positioned alongside the housing at a distance from the distal end of the housing, the foreign object may be positioned generally parallel to an elongation direction of the housing. This positioning of the foreign object may reduce the chance of the foreign object becoming stuck in the passage.

The first object manipulation tool may be fully or at least partially positioned or positionable inside the housing. The second object manipulation tool may be fully or at least partially positioned or positionable inside the housing, or may in embodiment be positioned outside of the housing. The second object manipulation tool may conceivably be positioned outside the housing, but may still be connected to the housing.

In general, an object manipulation tool is arranged for clamping, grasping, lassoing, looping around, hooking, or otherwise connecting to the foreign object. Any object manipulation tool may hence be embodied as a snare with a looped distal end, a clamp, forceps, or any other clamping, grasping, or lassoing tool. In general, when an object manipulation tool has been connected to a foreign object, a position of said object manipulation tool generally corresponds to a position of the part of the foreign object at which the object manipulation tool has been connected, thus allowing manipulation of a position and/or orientation of the foreign object using the object manipulation tool.

When the housing comprises a slit, extending from a distal end of the housing towards a proximal end of the housing, at least part of the first object manipulation tool may extend through the slit. This may allow the distal end of the first object manipulation tool to be positioned alongside the housing at a distance from the distal end of the housing. Part of the first object manipulation tool may in use be moved through the slit, in particular in a direction away from the distal end of the housing.

When the housing comprises a separator positioned inside the housing, two compartments may be formed inside the housing. The first object manipulation tool may be positioned in one of the compartments, and the second manipulation tool may be positioned in another of the compartments. This may prevent contact between the manipulation tools, and for example may prevent entanglement of manipulation tools or excess friction between manipulation tools.

When an object manipulation tool comprises a snare, or consists of a snare, said manipulation tool may have a loop-forming distal end. Optionally, the loop formed by the loop-forming distal end may be loosened and/or tightened by an operator, for example for grasping the foreign object more tightly. A snare typically has a low stiffness against bending and compression. As such, it may be difficult for an operator to position a foreign object into a loop of a snare. The housing of the device may in use provide sufficient stiffness in order to position the loop of the snare such that the foreign body can be moved into the loop. In general, the housing of the device may be formed as a flexible tube.

Any opening of any loop formed by any loop-forming distal end disclosed herein is preferably essentially free of obstructions, or even entirely free of obstructions. Any obstructions in the opening of the loop may for example make it more difficult to pass the foreign object through the loop. Examples of obstructions are lines or webbings for example forming a net to catch objects with.

Any object manipulation tool of any device disclosed herein may consist of a snare.

A second aspect provides an endoscopic assembly for retrieving a foreign object through an elongated passage. The assembly comprises a device according to the first aspect and an endoscopic camera with an elongated tube and a working channel through said tube, wherein the device is arranged to be inserted into the working channel.

The housing of the device may be separate from the elongated tube of the endoscopic camera, or may in embodiments be formed by the elongated tube of the endoscopic camera. In the latter case, the elongated tube of the endoscopic camera may comprise a slit extending from the distal end of the tube towards a proximal end of the tube, which slit is arranged for having at least part of the first object manipulation tool extending therethrough.

A third aspect provides a method of retrieving a foreign object through an elongated passage, comprising steps of inserting at least part of a housing of a device for retrieving a foreign object into the elongated passage, grasping the foreign object with a first object manipulation tool comprised by the device, grasping the foreign object with a second object manipulation tool comprised by the device, moving a distal end of the first object manipulation tool alongside the housing at a distance from a distal end of the housing, in particular after grasping the foreign object with the first object manipulation tool such that the foreign object is positioned generally parallel to the housing, and retracting the device through the elongated passage together with the foreign object, with the foreign object positioned generally parallel to the housing.

By moving a distal end of the first object manipulation tool alongside the housing at a distance from a distal end of the housing after grasping the foreign object with the first object manipulation tool, the foreign object may be positioned generally parallel to the housing. This in turn may reduce the chance of the foreign object becoming stuck in the passage.

In general, any device according the first aspect may be used in any embodiment of the method according to the second aspect. The method may be performed on a foreign object which is not present in a human or animal body. Alternatively, the foreign object is present in a human or animal body, and the elongated passage may be a body lumen of a human or animal, such as an oesophagus, trachea or rectum.

The foreign object may be an elongated foreign object, with a length larger than a width or diameter of at least one cross-section of the narrow passage, in particular at least 1.5 times larger or even 2 times larger or more. When the foreign object is positioned generally parallel to the housing, a distal end of the first object manipulation tool may be oriented at an angle relative to the housing, in particular generally perpendicular to an elongation direction of the housing.

When the foreign object is positioned generally parallel to the housing, a distal end of the second object manipulation tool may be oriented at an angle relative to the housing, in particular generally perpendicular to an elongation direction of the housing.

The distal end of one or both object manipulation tools may be oriented generally perpendicular to the foreign object when the foreign object is connected to the one or both object manipulation tools, in particular while the foreign object is moved through the elongated passage.

A distal end of an object manipulation tool may be oriented at an angle relative to the housing for example by virtue of the distal end of the object manipulation tool being bent or rotated. For example when an object manipulation tool comprises a snare, the snare may be bent over the housing.

It will be understood that options disclosed herein in conjunction with the device may be readily combined for different embodiments of the device. Furthermore, options disclosed herein in conjunction with the method may be readily combined for different embodiments of the method. Even, options disclosed herein in conjunction with the device may be readily applied in embodiments of the method, and vice versa.

### BRIEF DESCRIPTION OF THE FIGURES

In the figures,

FIGS. 1A-1C schematically show a distal end of an endoscopic tool;

FIGS. 2A-2D schematically depict consecutive steps in a method of retrieving a foreign object through an elongated passage;

FIGS. 3A-3B schematically depict consecutive steps in another method of retrieving a foreign object through an elongated passage; and FIG. 4A schematically shows an endoscopic assembly;

FIG. 4A schematically shows an endoscopic assembly.

FIG. 4B schematically shows a detailed view of part of FIG. 4A; and

FIG. 4C schematically shows a cross-sectional view of part of FIG. 4A.

### DETAILED DESCRIPTION OF THE FIGURES

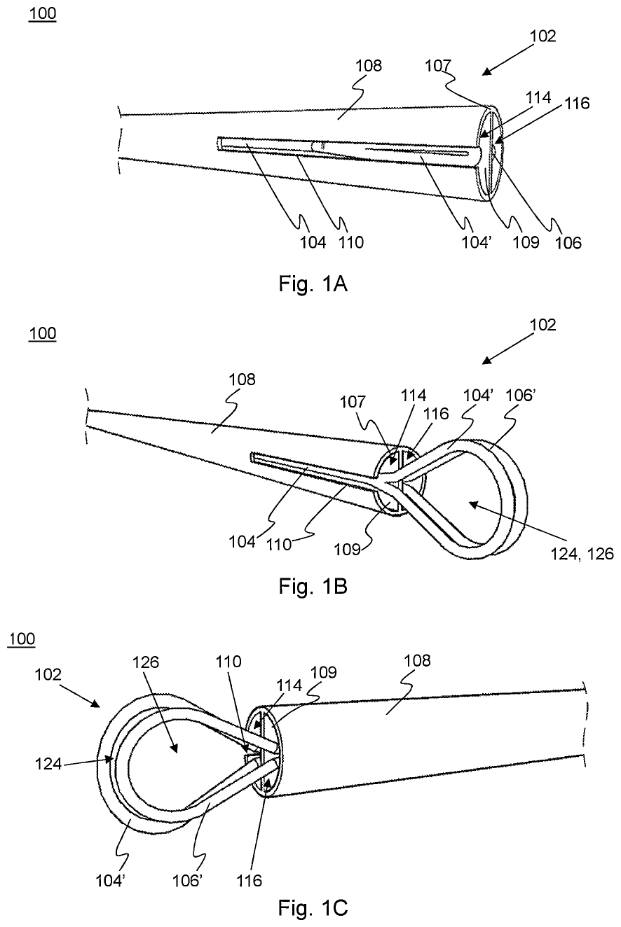

FIG. 1A schematically shows a distal end 102 of an endoscopic tool 100, arranged for retrieving a foreign object through an elongated passage. It will be understood that throughout the figures, the device 100 is only partially shown for conciseness of the figures. Thus, the device 100 may have any length beyond the distal end 102 of the device 100 depicted in the figures. At a proximal end of the device 100, not depicted for conciseness of the figures, one or more handles and/or manipulators may be present, allowing an operator of the device 100 to control a position and/or orientation of the device, and/or allowing an operator of the device 100 to control one or more manipulation tools comprised by the device 100—in particular from outside the passage into which the device 100 can be inserted.

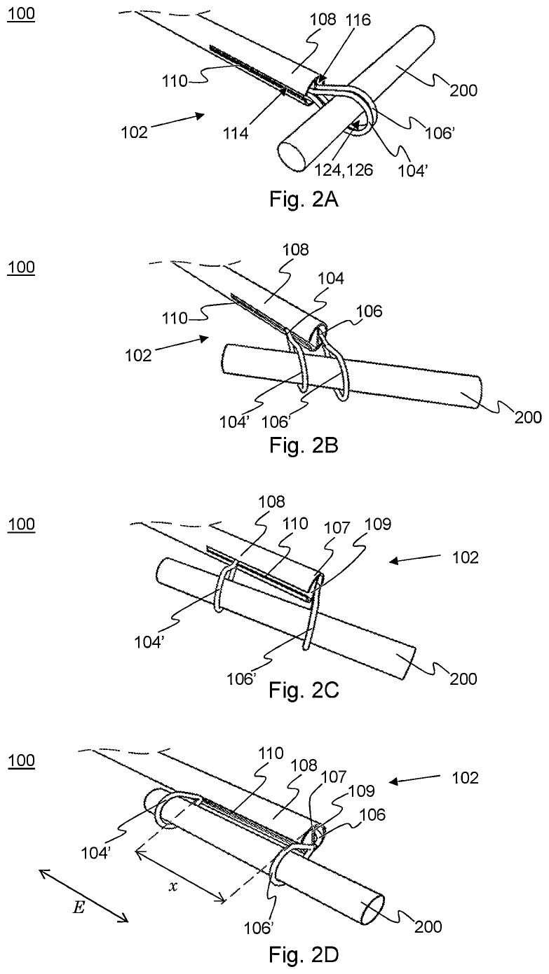

FIG. 1A shows the device 100 comprising two manipulation tools 104, 106, which at least in FIGS. 1A-2D are embodied as snares with a loopforming distal end 104′, 106′. In FIG. 1A, the snares 104, 106 are contained within a housing 108 of the device. In FIGS. 1B and 1C, two isometric views of the device 100 are depicted, wherein distal ends, in particular loop-forming distal ends 104′, 106′, of the snares 104, 106 extend out of the housing 108. It will be understood that in other embodiments, one or both of the manipulation tools may be embodied as any other option of an object manipulation tool disclosed herein, in any combination thereof.

In the state of FIG. 1A, the loop-forming distal ends 104′, 106′ of the snares 104, 106 are contained within the housing 108. As such, an outer footprint of the distal end 102 of the device 100 may be essentially determined by an outer footprint of the housing 108—for example by an outer diameter of the housing 108. The outer diameter of the housing 108 may be smaller than or generally correspond to a diameter of a passage into which the housing 108 is to be inserted. It will be understood that a cross-section of a passage may be elastically deformed when the housing 108 is inserted into said passage for accommodating the housing 108 in the passage. In the state of FIG. 1A, the snares 104, 106 thus do not extend out of a distal end 107 of the housing.

In the state depicted for example in FIGS. 1B and 1C, the loopforming distal ends 104′, 106′ have been moved out of the housing 108, for example by virtue of a manual operation of an operator at or near a proximal end of the device 100 (not shown in the figures). As the loop-forming distal ends 104′, 106′ have moved out of the housing 108, a first loop 124 is formed by the loop-forming distal end 104′ of the first snare 104, and a second loop 126 is formed by the loop-forming distal end 106′ of the second snare 106. In other words, the loop-forming distal ends have become deployed.

As a general option shown for example in FIGS. 1B and 1C, an outer footprint of the loops 124, 126 when deployed may be larger than an outer footprint of the housing 108. In other words, parts of the loop 124, 126 may extend beyond an outer footprint of the housing 108. During insertion of the device into the passage, it may thus be preferred to contain the loop forming distal ends 104′, 106′ at least partially but preferably fully inside the housing, in particular to minimize the outer footprint of the device 100.

As can be seen in the figures, for example in FIG. 1B, as a preferred feature for any loop disclosed herein, the loop defines an opening that is essentially free of obstructions.

As an option depicted in FIGS. 1A-1C, the housing 108 comprises a slit 110 extending from a distal end of the housing 108 towards the proximal end of the housing 108. The slit 110 is dimensioned to allow at least part of the first snare 104 to pass through the slit 110 and out of the housing 108. In particular, a section of the first snare 104 adjacent to the loop-forming section 124′ can pass in a generally radial direction through the slit 110 and out of the housing 108—as will be elaborated on in conjunction with FIGS. 2A-2D.

Furthermore, depicted in FIGS. 1A-1C, is an option for the housing 108 wherein the housing 108 comprises a separator 109 positioned inside the housing 108. By virtue of the separator 109, two separate compartments 114, 116 are obtained in the housing 108. The separator 109 may at least partially prevent the two snares 104, 106 from contacting inside the housing and/or from becoming constricted inside the housing.

As a particular option shown in FIG. 1C, the loops 124 and 126 may be sized and/or shaped differently. This may allow an operator to distinguish the two loops 124, 126 from each other. Loops being sized and/or shaped differently here implies that a surface area through the loops is different, and/or that the surface area through the loops are shaped differently.

In particular, the loop 126 formed by the loop-forming distal end 106′ of the second object manipulation tool 106 may be smaller than the loop 124 formed by the loop-forming distal end 104′ of the first object manipulation tool 104. This may allow the object 200 to be positioned more easily inside the loop 126 formed by the loop-forming distal end 106′ of the second object manipulation tool 106.

It will be understood that different options disclosed herein, for example in conjunction with the housing, may be readily combined into different embodiments of the device 100.

FIGS. 2A-2D schematically depict consecutive steps in a method of retrieving a foreign object 200 through an elongated passage. The method may be generally applied in a medical field, but also in a non-medical field. Although in the figures the foreign object 200 is depicted as a cylinder, it will be understood that any shape of foreign object may be retrieved using the device. In particular, a foreign object may be an elongated object, which may have a length exceeding a diameter of the passage through which the foreign object has to be retrieved. In other words, it may be required to position the foreign object generally parallel to the passage to prevent the object from becoming stuck or jammed in the passage.

In the embodiment of the device 100 shown in FIG. 2A-2D, the first object manipulation tool and the second object manipulation tool are embodied as snares 104, 106 with a loop-forming distal end 104′, 106′.

FIG. 2A shows a step in a method of retrieving a foreign object through an elongated passage, wherein the foreign object 200 passes through two loops 124, 126 formed by two snares 104, 106—which snares are examples of manipulation tools. Optionally, one or both loops 124, 126 may be tightened to increase grip on the foreign object 200. A loop can for example be tightened by retracting the snare at one end of the loop.

In FIG. 2A, the two snares 104, 106 are depicted in a generally parallel orientation relative to each other. It will be appreciated that in any method disclosed herein, preferably, the first and second object manipulation tools are oriented generally parallel while inserting the housing into the elongated passage.

In FIG. 2A, as a preference for any device and method disclosed herein, the openings of the loops 124, 126 are at least partially aligned at least before the object 200 passes through the loops 124, 126. As such, in a single movement of the object 200, the object 200 can be positioned through both loops.

FIG. 2B shows a consecutive step, wherein the first snare 104 is moved into the slit 110 of the housing 108. Already by virtue of this movement, the object 200 is manipulated from an orientation generally perpendicular to an elongation direction of the housing 108 (shown in FIG. 2A) to an orientation more parallel to the elongation direction of the housing 108.

As can be seen in FIG. 2B, the first snare 104, in particular the distal end 104′, is being moved alongside and outside the housing 108. Preferably, the first snare 104 is moved into the slit after inserting the housing into the elongated passage. In general, in the present disclosure, generally parallel to the elongation direction of the housing implies less than 45 degrees offset from the elongation direction of the housing, less than 30 degrees offset, less than 15 degrees offset, or even less than 5 degrees offset from the elongation direction of the housing.

FIG. 2C shows an even further step, wherein the first snare 104 is moved through the slit 110 further away from the distal end 107 of the housing 108. This movement of the first snare 104, and the fact that the object 200 is held in the first loop-forming distal end 104′, causes the object 200 to be positioned even more parallel to the elongation direction of the housing 108. The second snare 106 becomes bent, which causes the distal end of the second snare 106 to become further oriented radially relative to the elongation direction of the housing 108, for example by being bent over the housing 108, in particular over the optional separator 109 in the housing.

In FIG. 2D, the first snare 104 has been fully moved through the slit 110, and the loop-forming distal end 104′ is positioned at a distance x from the distal end 107 of the housing 108. The object 200 is now positioned essentially parallel to the elongation direction E of the housing 108, and can be moved together with the housing 108 through a narrow passage with less chance of becoming stuck in said passage. After the first snare 104 has been fully moved through the slit 110, the first snare 104 can abut the housing at the end of the slit.

In general, the distance x may be larger than 10 mm, larger than 20 mm, larger than 30 mm—for example between 30 mm and 50 mm—or even larger than 50 mm. The distance x may depend on the size, in particular the length, of the foreign object. In general, a larger foreign object may require a larger distance x. For example, when the length of the foreign object exceeds 50 mm, the distance x may be 30 mm or more. When the length of the foreign object is below 50 mm, the distance x may be 30 mm or less, for example about 20 mm. As generally depicted in FIGS. 1A-2D, as options optionally applicable to any device disclosed herein, the two compartments 114, 116 may be generally of the same length and/or the distal ends of the compartments 114, 116 are at least approximately aligned.

Additionally, or alternatively, the housing only has a single slit 110 at the distal end 102 of the housing. Even further additionally or alternatively, only one of the compartments of the housing has a slit 110 therein.

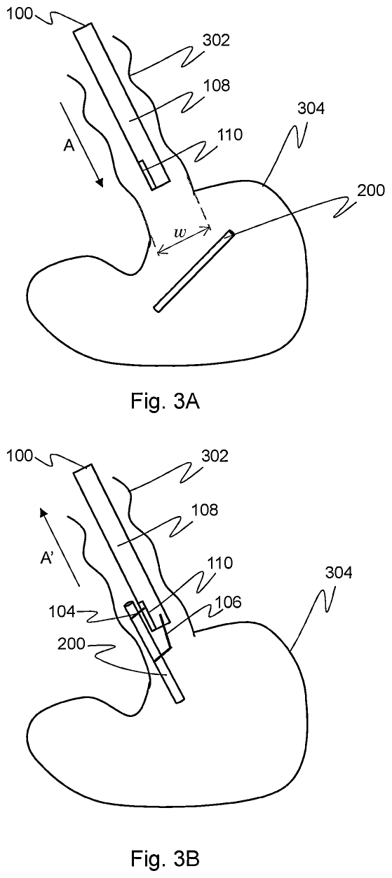

FIGS. 3A-3B schematically show two consecutive steps in a method of retrieving a foreign object 200 from a stomach 304, through an oesophagus 302 as an example of an elongated passage, which may be a human or animal oesophagus. It will be understood that FIGS. 3A-3B merely serve as a schematic example, and many different situations are envisioned in which the endoscopic tool 100 can be used for retrieving a foreign object through an elongated passage, also in non-medical examples or other medical examples, wherein the passage can for example be any part of the gastro-intestinal tract or respiratory tract of a human or animal.

In the situation depicted schematically in FIG. 3A, an embodiment of a device 100 has been inserted in the oesophagus 302, in a direction generally indicated with arrow A. The device 100 is generally inserted towards the foreign object 200, which in this particular example is generally positioned inside the stomach 304.

As can be seen in FIG. 3A, the elongated passage, in this case an oesophagus 302, may have a width w which is smaller than a length of the foreign object 200. In particular in these cases, a device of the first aspect and a method of the second aspect advantageously allow the foreign object 200 to be positioned generally parallel to the housing 108. In this orientation, the length of the foreign object 200 is oriented generally in a direction parallel to the elongated passage, and hence the foreign object is less likely to become stuck in the elongated passage. In the situation schematically depicted in FIG. 3B, the foreign object 200 has been engaged by the device 100. In particular, the object 200 has been engaged by a first manipulation tool 104 and a second manipulation tool 106. In general, when an object is engaged by an object manipulation tool, the manipulation tool may be used for moving the object, for example in a translational movement and/or a rotation.

With the foreign object 200 engaged by the device 100, the foreign object can be retracted through the elongated passage together with the device. Preferably, for any method disclosed herein, the device may be retracted through the elongated passage together with the foreign object, with the foreign object positioned outside the housing.

In general, an object manipulation tool may be a snare with a loopforming section, a clamping or grasping device, such as a pair of forceps, tweezers, clips, clamps, tongs or pliers, a hooking tool with a hook at a distal end thereof, a suction tool for forming a vacuum connection with an object, a magnetic tool for forming a magnetic connection with an object, or any other tool arranged for connecting with an object such that the object can be manipulated. In FIG. 3A, the manipulation tools 104, 106 are only depicted in a schematic way, and it will be understood that any embodiment of the device 100 disclosed herein is envisioned with any first manipulation tool, any second manipulation tool, in any combination.

By virtue of the endoscopic tool 100 comprising two manipulation tools 104, 106, the object 200 may be engaged at two different locations on the object 200, which locations are spaced apart. This may allow better control for an operator of the endoscopic tool in rotating and/or translating the object 200. In particular, when the object 200 has to be rotated, for example in order to position the object 200 more parallel to the housing 108 of the device, engaging the object 200 with two manipulation tools 104, 106 may make this rotation possible or more convenient for the operator. As can be seen in FIG. 3B, the housing 108 of the endoscopic tool 100 comprises a slit 110 therein. In the state of FIG. 3B, after the object 200 has been positioned generally parallel to the housing 108, the first manipulation tool 104 extends through the slit 110 and is oriented generally perpendicular to the housing 108. In the present disclosure, generally perpendicular may be between 30 degrees and 150 degrees relative to an elongation direction of the housing, or even between 60 degrees and 120 degrees, or even approximal 90 degrees relative to an elongation direction of the housing.

As can further be seen in FIG. 3B is that a distal end of the second manipulation tool 106 may also be oriented generally perpendicular relative to an elongation direction of the housing. This may allow the object 200 to be positioned even more parallel to the housing 108 of the device 100.

In the state of the device depicted in FIG. 3B, the first manipulation tool 104 is positioned alongside and outside the housing at a distance) from the distal end of the housing.

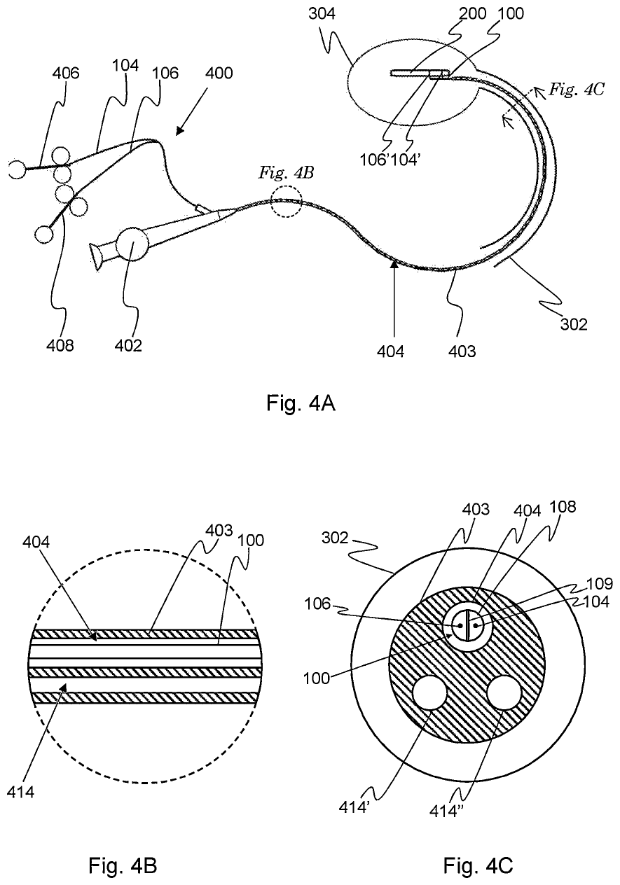

FIG. 4A schematically shows an endoscopic assembly 400 which is partially inserted into a body passage 302. The endoscopic assembly 400 comprises an endoscopic camera 402 with an elongated tube 403 and a working channel 404 through said tube. An embodiment of the endoscopic tool 100 extends through the working channel 404. It will be understood that the housing 108 of the device 100 may in examples be formed by part of the tube 403 of the endoscopic camera 402, in particular by a distal end of said tube 403.

In FIG. 4A, the device 100 is shown comprising the two snares 104, 106 as manipulation tools. The snares 104, 106 extend through the working channel 404 between a proximal end at which two manipulators 406, 408 are provided, and a distal end which in use may be positioned inside a stomach 304 or any other remote location accessible via the elongated passage 302. The manipulators 406, 408 may for example be used to push the snares further out of the tube 403 and/or to tighten or loosen loops formed by loopforming distal ends 104′, 106′ of the snares 104, 106.

Any snare may loop back towards their respective manipulator. As such, both ends of any snare may be positioned at or near one of the manipulators. For example by retracting one of the ends, a loop formed by the snare at the distal end of the device may be tightened.

FIG. 4B schematically shows a detailed section view of part of the tube 403 of the endoscopic camera 402. As can be seen, the tube 403 comprises a working channel 404, which may also be referred to as a biopsy channel. The device 100 in use extends through this working channel 404 towards the foreign object 200. As an example, FIG. 4B shows the tube 403 further comprising a camera channel 414 therethrough, for example through which one or more optical fibres of the endoscopic camera 402 may extend. It will be understood that in general, any number of channels may run through the tube 403, for example for as a light guide for the camera but also for transporting one or more fluids such as water of air through the tube 403.

FIG. 4C schematically shows a cross-sectional view of part of FIG. 4A. As can be seen from FIG. 4C, the device 100 is inserted into the working channel 404. Preferably, the housing 108 of the device 100 contains the first manipulation tool 104 and the second manipulation tool 106. Optionally, the separator 109 is provided to form separate compartments for the two manipulation tools. Also indicated in FIG. 4C are two further channels 414′ 414″, which for example may be used for the camera 402 and/or for transporting fluid through.

When the device 100 is retracted through the passage together with the object 200, the object may remain outside the housing of the device, and also outside the tube 403.

Although the examples in the figures may refer to a medical application of the device and method disclosed herein, it will be appreciated that the same device and method may be used for non-medical application—even without modification of the device and method of using the device.

In the description above, it will be understood that when an element is referred to as being connected to another element, the element is either directly connected to the other element, or intervening elements may also be present. Also, it will be understood that the values given in the description above, are given by way of example and that other values may be possible and/or may be strived for.

It is to be noted that the figures are only schematic representations of embodiments that are given by way of non-limiting examples. For the purpose of clarity and a concise description, features are described herein as part of the same or separate embodiments, however, it will be appreciated that the scope of the disclosure may include embodiments having combinations of all or some of the features described.

The word ‘comprising’ does not exclude the presence of other features or steps. Furthermore, the words ‘a’ and ‘an’ shall not be construed as limited to ‘only one’, but instead are used to mean ‘at least one’, and do not exclude a plurality.

In summary, a device and method are provided for retrieving a foreign object through an elongated passage. The device comprises a first object manipulation tool, a second object manipulation tool, an elongated housing arranged for accommodating at least part of the first object manipulation tool and at least part of the second object manipulation tool. The first object manipulation tool and the second object manipulation tool are arranged to be partially moved out of the housing at or near a distal end of the housing, and the first object manipulation tool is arranged to have a distal end of the first object manipulation tool positioned alongside the housing at a distance from the distal end of the housing. Using the at least two object manipulation tools allows the foreign object to be grasped or clamped at two distinct locations. By positioning the first object manipulation tool alongside the housing after the object has been grasped or clamped, the object can be positioned approximately parallel to the housing, and may as such be less likely to get stuck in said passage, and/or it is less likely that the object becomes disconnected from the device when retracting the object out of the elongated passage. The device may be inserted through a working channel of an endoscopic camera.

1-29. (canceled)

30. A device for retrieving a foreign object through an elongated passage, the device comprising:
a first object manipulation tool;
a second object manipulation tool;
an elongated housing, such as a flexible tube, arranged for accommodating at least part of the first object manipulation tool, and preferably also at least part of the second object manipulation tool,
wherein the first object manipulation tool, and preferably also the second object manipulation tool, is arranged to be partially moved out of the housing at or near a distal end of the housing;
and the first object manipulation tool is arranged to have a distal end of the first object manipulation tool positioned alongside the housing at a distance (x) from the distal end of the housing.

31. The device of claim 30, wherein the first object manipulation tool is arranged to have a distal end of the first object manipulation tool positioned alongside and outside the housing at the distance from the distal end of the housing.

32. The device of claim 30, wherein the housing comprises a slit extending from the distal end of the housing towards a proximal end of the housing, which slit is arranged for having at least part of the first object manipulation tool extending therethrough.

33. The device of claim 30, wherein the housing comprises a separator positioned inside the housing, which separator forms two separate compartments in the housing, and wherein the first object manipulation tool is positioned in one of the compartments, and the second manipulation tool is positioned in another of the compartments.

34. The device of claim 30, wherein the housing comprises a slit extending from the distal end of the housing towards a proximal end of the housing, which slit is arranged for having at least part of the first object manipulation tool extending therethrough and wherein the housing comprises a separator positioned inside the housing, which separator forms two separate compartments in the housing, and wherein the first object manipulation tool is positioned in one of the compartments, and the second manipulation tool is positioned in another of the compartments.

35. The device of claim 30, wherein the first object manipulation tool comprises a snare, with a loop-forming distal end.

36. The device of claim 35, wherein the loop-forming distal end of the snare of the first object manipulation tool forms a loop which defines an opening that is essentially free of obstructions.

37. The device according to claim 30, wherein the second object manipulation tool comprises a snare, with a loop-forming distal end.

38. The device of claim 37, wherein the loop-forming distal end of the snare of the second object manipulation tool forms a loop which defines an opening that is essentially free of obstructions.

39. The device of claim 30, wherein the first object manipulation tool comprises a snare, with a loop-forming distal end, wherein the loop-forming distal end of the snare of the first object manipulation tool forms a loop which defines an opening that is essentially free of obstructions.

40. The device of claim 37, wherein a loop formed by the loop-forming distal end of the first object manipulation tool is sized and/or shaped different than a loop formed by the loop-forming distal end of the second object manipulation tool, in particular wherein the loop formed by the loop-forming distal end of the second object manipulation tool is smaller than the loop formed by the loopforming distal end of the first object manipulation tool.

41. The device of claim 35, wherein the loop formed by the snare of the first object manipulation tool can be tightened.

42. The device of claim 37, wherein the loop formed by the snare of the second object manipulation tool can be tightened.

43. The device of claim 30, wherein the elongated housing is arranged to be inserted into a working channel of an endoscopic camera.

44. An endoscopic assembly for retrieving a foreign object through an elongated passage, comprising: an endoscopic tool; and an endoscopic camera with an elongated tube and a working channel through said tube, wherein the device is arranged to be inserted into the working channel.

45. A method of retrieving a foreign object through an elongated passage, comprising the steps of:
inserting at least part of a housing of a device for retrieving a foreign object through an elongated passage into the elongated passage;
grasping the foreign object with a first object manipulation tool comprised by the device;
grasping the foreign object with a second object manipulation tool comprised by the device;
moving a distal end of the first object manipulation tool alongside the housing at a distance from a distal end of the housing, in particular after grasping the foreign object with the first object manipulation tool, such that the foreign object is positioned generally parallel to the housing; and
retracting the device through the elongated passage together with the foreign object, with the foreign object positioned generally parallel to the housing.
